(19) United States
(12) Patent Application Publication
(10) Pub. No.: US 2024/0127960 A1
(43) Pub. Date: Apr. 18, 2024

(54) DASHBOARD FOR TRACKING HEALTHY BUILDING PERFORMANCE

(71) Applicant: Honeywell International Inc., Charlotte, NC (US)

(72) Inventors: Rajkumar Palanivel, Eden Prairie, MN (US); Himanshu Khurana, Excelsior, MN (US); Deenadayalan Karunakaran, Bangalore (IN); Chandrakantha Chandra, Charlotte, NC (US); Hemant Jagannath Wadnere, Pune (IN); Ganapati Hegde, Charlotte, NC (US); Naveen Kumar Dindukurthi Sivaprasad, Bangalore (IN); Chen Xu, Shanghai (CN)

(21) Appl. No.: 18/391,312

(22) Filed: Dec. 20, 2023

Related U.S. Application Data
(63) Continuation of application No. 17/141,844, filed on Jan. 5, 2021, now Pat. No. 11,894,145.
(60) Provisional application No. 63/085,921, filed on Sep. 30, 2020.

(51) Int. Cl. G16H 50/30; F24F 11/63; G05B 15/02; G08B 21/02; G16H 15/00; G16H 80/00

(52) U.S. Cl. CPC G16H 50/30; F24F 11/63; G05B 15/02; G08B 21/02; G16H 15/00; G16H 80/00; F24F 2110/20

(57) ABSTRACT

A method of monitoring building compliance with healthy building guidelines includes obtaining current parameter values for a plurality of different parameters from a plurality of sensors disposed within a plurality of different zones of a building. For each of the parameters, the current parameter value received from each of the plurality of different zones is compared with a corresponding healthy building range for that parameter as specified by the healthy building guidelines. A healthy building dashboard is displayed that includes a summary that shows, for each of the plurality of different parameters, how many zones of the plurality of different zones of the building are within the corresponding healthy building range for that parameter and/or how many zones of the plurality of different zones are not within the corresponding healthy building range for that parameter.

### RELATED APPLICATION

This is a continuation of co-pending U.S. patent application Ser. No. 17/141,844, filed Jan. 5, 2021, which claims the benefit of U.S. Provisional Patent Application Ser. No. 63/085,921, filed Sep. 30, 2020, both of which are incorporated herein by reference.

### TECHNICAL FIELD

The present disclosure pertains to monitoring building performance. More particularly, the present disclosure pertains to monitoring compliance with healthy building standards.

### BACKGROUND

Coronavirus disease 2019 (COVID-19) is an infectious disease caused by severe acute respiratory syndrome coronavirus 2 (SARS-CoV-2). COVID-19 is one of many infectious diseases transmitted via airborne and/or other particles. In some cases, it may be difficult to mitigate the spread of infectious diseases, particularly diseases transmitted through airborne and/or other particles, at indoor facilities (e.g., buildings, department stores, warehouses, plants, factories, refineries, airports, laboratories, school buildings, theaters, etc.) due to the indoor environment, proximity of occupants, and/or other factors. Often, these indoor facilities have various building automation systems (e.g., heating, ventilation, and air conditioning (HVAC) systems, surveillance systems, security systems, energy management systems, etc.) to control environmental conditions of the indoor facility and/or monitor occupancy. A need remains for ways to monitor how well a building or other facility is performing with respect to meeting healthy building guidelines.

### SUMMARY

The present disclosure relates to a monitoring compliance with prescribed guidance, and more particularly, to monitoring compliance of a facility such as a building with healthy building guidelines that are intended to reduce changes of infectious disease spread within the facility.

In an example, a method of monitoring building compliance with healthy building guidelines, where the healthy building guidelines specify desired ranges for each of a plurality of different parameters, includes obtaining current parameter values for a plurality of different parameters from a plurality of sensors disposed within a plurality of different zones of a building. For each of the parameters, the current parameter value received from each of the plurality of different zones is compared with a corresponding healthy building range for that parameter as specified by the healthy building guidelines. A healthy building dashboard is displayed that includes a summary that shows, for each of the plurality of different parameters, how many zones of the plurality of different zones of the building are within the corresponding healthy building range for that parameter and/or how many zones of the plurality of different zones are not within the corresponding healthy building range for that parameter.

In another example, a method of monitoring building compliance with healthy building guidelines, where the healthy building guidelines specify desired ranges for each of a plurality of different parameters, includes receiving parameter values for a plurality of different parameters in a building. The current values for each of the plurality of different parameters are compared to a corresponding healthy building range specified in the healthy building guidelines to help reduce the spread of disease in the building. A healthy building dashboard is displayed on a display that indicates for each of the plurality of different parameters whether any areas of the building are not within the healthy building range for that parameter. In response to a user request, additional information is displayed on the display that pertains to the areas of the building for which one or more of the parameters are outside of the healthy building range for that parameter.

In another example, a non-transient, computer-readable storage medium stores instructions that when executed by a processor cause the processor to receive parameter values for a plurality of different parameters in a building over time, compare current values for each of the plurality of different parameters to a healthy building range specified for each of the plurality of different parameters and display a dashboard on a display that indicates for each of the plurality of different parameters whether any areas of the building are not within the healthy building range for that parameter. In response to a user request, additional information may be displayed on the display that pertains to the areas of the building for which one or more of the parameters are outside of the healthy building range for that parameter.

The preceding summary is provided to facilitate an understanding of some of the features of the present disclosure and is not intended to be a full description. A full appreciation of the disclosure can be gained by taking the entire specification, claims, drawings, and abstract as a whole.

### BRIEF DESCRIPTION OF THE DRAWINGS

The disclosure may be more completely understood in consideration of the following description of various illustrative embodiments of the disclosure in connection with the accompanying drawings, in which:

FIG. 1 is a schematic view of an illustrative system for monitoring building compliance with healthy building guidelines;

FIG. 2 is a flow diagram showing an illustrative method of monitoring building compliance with healthy building guidelines;

FIG. 3 is a flow diagram showing an illustrative method of monitoring building compliance with healthy building guidelines; and

FIGS. 4 through 21 are screen captures showing illustrative screens that may be displayed by the illustrative system of FIG. 1.

While the disclosure is amenable to various modifications and alternative forms, specifics thereof have been shown by way of example in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings wherein like reference numerals indicate like elements. The drawings, which are not necessarily to scale, are not intended to limit the scope of the disclosure. In some of the figures, elements not believed necessary to an understanding of relationships among illustrated components may have been omitted for clarity.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a” “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

Facilities often include building automation systems (e.g., heating, ventilation, and air conditioning (HVAC) systems, surveillance systems, security systems, energy management systems, etc.). Various organizations worldwide (e.g., government organizations, educational organizations, etc.) have provided guidelines on how to operate building automation system to reduce risk of disease transmissions within facilities. Similarly, various organizations worldwide have provided guidelines on how occupants of a facility and monitoring occupancy can reduced risk of disease transmission. Other guidelines relating to facilities and transmission of infectious disease are contemplated and may be adapted and used, depending on the facility.

It can be difficult for facility managers to assess performance of their facilities, occupants, etc. against guidance (e.g. guidelines, rules, etc.). In some cases, the guidance may specify desired environmental conditions and desired occupancy/occupant behavior to help reduce or mitigate risk of disease transmission in a facility. Additionally, in view of various guidelines from various organizations, it has been difficult for facility managers to assess whether their facilities have the necessary sensor devices and/or other suitable sensing or monitoring equipment to accurately judge and/or reduce the risk of disease transmission.

This disclosure provides methods and systems for assessing a facilities compliance with various guidelines related to reducing risk of infectious disease (e.g., COVID-19, Ebola, influenza, common cold, airborne diseases, and/or other infectious diseases) transmissions. Sensing devices of existing building automation systems and/or other suitable sensing devices may be utilized. In some cases, methods and systems are provided to help facility managers identify when additional sensors, cameras, and/or other equipment may help in mitigating risk of transmitting infectious diseases in their facility, and in some cases, may quantify how much such additional equipment may help mitigate the risk of transmission of infectious diseases in their facility.

The techniques and/or systems disclosed herein may provide displays offering facility managers easily understandable performance metrics of a facility and/or facility occupants' against a set of infectious disease guidelines. The metrics may incorporate and/or be based on knowledge of a facility location (e.g., geographic location), knowledge of a facility size, knowledge of a facility floorplan, knowledge of sensing devices at a facility, knowledge of common HVAC system capability, and parameters, adjustable values or weights that may be tuned based on climate, building characteristics, and evolving knowledge (e.g., guidelines, studies, laws, etc.) related to disease transmission, etc.

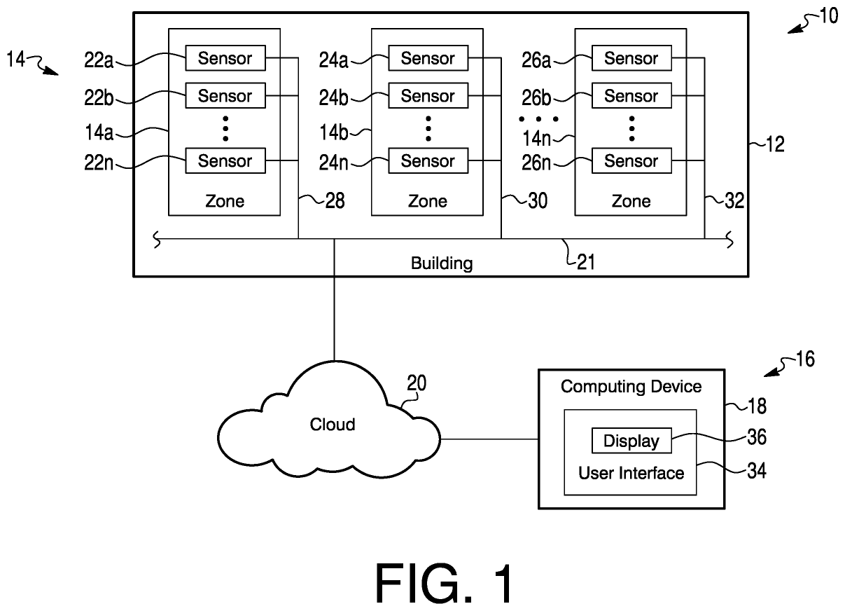

FIG. 1 is a schematic block diagram of an illustrative system 10 for monitoring building compliance with healthy building guidelines. Healthy building guidelines may, for example, include infectious disease guidance. Infectious disease guidance may include recommendations related to values, counts, percentages, and/or other measures of one or more of relative humidity in a facility, CO₂ concentration in a facility, air change rates in a facility, occupancy in a facility, particulate matter concentrations in a facility, total volatile organic compound (TVOC) concentrations in a facility, a maximum occupancy level in a facility or a zone in a facility, maximum occupancy density in a facility, a number of crowd incidents per day in the facility, a percent of health related standard operating procedure actions that have been closed by an facility operator, mask compliance in a facility, elevated body temperature incidents in the facility, number of people potentially exposed to an infected individual in the facility determined via contact tracing, and/or recommendations or actions related to one or more other suitable factors affecting disease transmission within a facility.

The illustrative system 10 of FIG. 1 includes a building 12. It will be appreciated that the building 12 may represent a single building, or a collection of buildings. In some instances, the building 12 may represent a portion of a facility. Illustrative but non-limiting examples of buildings 12 include buildings, department stores, warehouses, plants, factories, refineries, airports, laboratories, office buildings, school buildings, theaters, arenas, stadiums, hotels, dorms, lecture halls, restaurants, etc. The building 12 may be considered as being divided into zones 14 that are individually labeled as 14a, 14b through 14n. Each zone 14 may represent a room or collection of rooms in the building 12. Each zone 14 may represent a floor of the building 12. Each zone 14 may represent particular regions within the building 12 that may not correspond directly to a particular room and/or a particular floor of the building 12. In some instances, the zones 14 may correspond to Heating, Ventilating and Air Conditioning (HVAC) system zones. In some cases, the zones 14 may represent divisions of the building 12 based upon use of various parts of the building 12. For example, a particular zone 14 may represent a collection of offices within the building 12 while another zone 14 may represent warehouse space. Another zone 14 may represent part or all of a parking garage, for example. It will be appreciated that in this, the ventilation and other needs of these various zones 14 may vary, sometimes considerably, based on how they are being utilized. Accordingly, it can make sense to define zones in accordance with how the various parts of the building 12 are being utilized.

The illustrative system 10 further includes a computing system 16. As illustrated, the computing system 16 includes a computing device 18 and one or more cloud servers 20. The building 12 includes a building network 21 that enables devices within the building 12 to communicate with the computing system 16, and in some cases with each other. It will be appreciated that in some instances the computing device 18 may be disposed within the building 12 and may itself be in communication with the building network 21 without requiring any intervening servers such as but not limited to the cloud server 20. The computing device 18 may itself be manifested within the cloud server 20. In some cases, the computing device 18 may be and/or may be part of, for instance, a smart phone, a tablet, a personal digital assistant (PDA), a personal computer, a beacon, a camera, a display device, a video recorder, a network component, a server, and/or other suitable computing device. In some cases, the computing device 18 may be distributed amount two or more devices.

As illustrated, the building 12 includes a number of sensors disposed within each of the zones 14. For example, the zone 14a includes a sensor 22a, a sensor 22b and through a sensor 22m. The zone 14b includes a sensor 24a, a sensor 24b and through a sensor 24m. The zone 14n includes a sensor 26a, a sensor 26b and through a sensor 26m. It will be appreciated that each of the sensors 22, 24, 26 may measure or detect any of a variety of different measures that are related to one or more parameters that may be part of the healthy building guidelines. The sensors 22, 24, 26 may include one or more of occupancy sensors, video cameras, still cameras, identification card readers, control signal monitors (e.g., to monitor when and/or how devices affecting infectious disease are used, such as UV lights, air exchangers, fans, etc.), air sensors, humidity sensors, temperature sensors, CO₂ (carbon dioxide) sensors, CO (carbon monoxide) sensors, thermostats, particulate matter sensors, TVOC (total volatile organic compound) sensors, thermometers, infrared sensors, pressure sensors (e.g., to monitor and/or effect pressure zones configured to exchange air in a specified zone in a facility), etc.

In the example show, the sensors 22a, 22b through 22m are able to communicate with the building network 21 and hence with the computing system 16 via a zone network 28. The sensors 24a, 24b through 24m are able to communicate with the building network 21 and hence with the computing system 16 via a zone network 30. The sensors 26a, 26b through 26m are able to communicate with the building network 21 and hence with the computing system 16 via a zone network 32. In some cases, signals from each of the sensors 22, 24, 26 are communicated to the computing device 18, either directly or via the cloud server 20. The computing device 18 includes a user interface 34 that may be used to provide instructions and other information to the computing device 18 and/or to receive information from the computing device 18. The user interface 34 may include any desired type of data entry equipment, such as but not limited to a keyboard, a mouse, a touch pad, a drawing pad and the like. The user interface 34 may include a display 36 that can be used to display information. The display 36 may include one or more separate monitors, each of the one or more separate monitors being addressable by the computing device 18.

In some cases, the display 36 may be used to display a dashboard that enables a user to quickly and easily ascertain how the building 12 is performing relative to various healthy building guidelines. Such a dashboard may display current values of various parameters measured or otherwise indicated by the sensors 22, 24, 26, and may include comparisons of the current values of those parameters with healthy building ranges for those parameters. Subsequent Figures will provide examples of dashboards that may be displayed on the display 36.

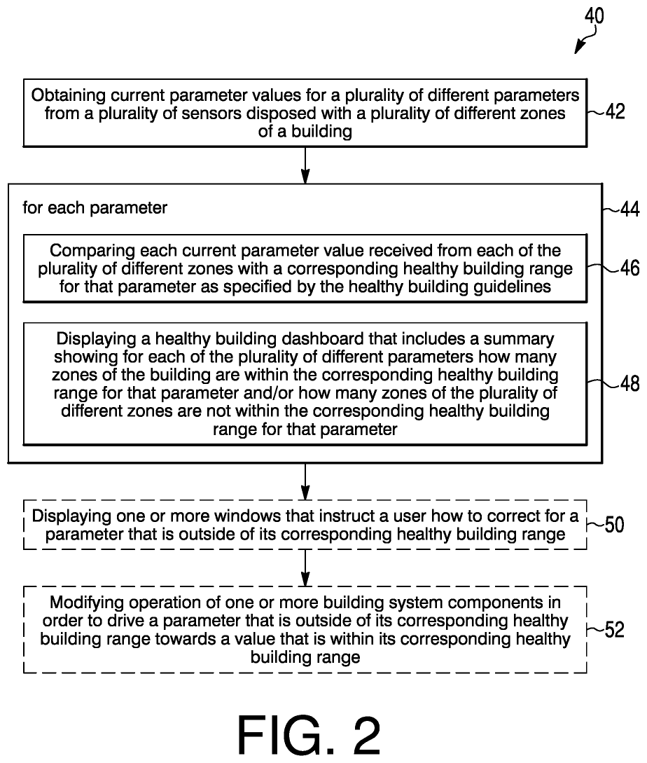

FIG. 2 is a flow diagram showing an illustrative method 40 of monitoring building compliance with healthy building guidelines, where the healthy building guidelines specify desired ranges for each of a plurality of different parameters. Current parameter values may be obtained for each of a plurality of different parameters from a plurality of sensors (such as the sensors 22, 24, 26) that are disposed within a plurality of different zones (such as the zones 14) of the building 12, as indicated at block 42. The values for each parameter may be processed, as indicated at block 44. In more detail, each current parameter value received from each of the plurality of different zones may be compared with a corresponding healthy building range for that parameter as specified by the healthy building guidelines, as indicated at block 46. A healthy building dashboard may be displayed that includes a summary that shows, for each of the plurality of different parameters, how many zones of the plurality of different zones of the building are within the corresponding healthy building range for that parameter and/or how many zones of the plurality of different zones are not within the corresponding healthy building range for that parameter, as indicated at block 48. In some cases, at least a region of the dashboard may collect and display only parameters that have a corresponding healthy building range specified by the healthy building guidelines. In some cases, all available parameters that have a corresponding healthy building range specified by the healthy building guidelines are displayed in this region. When so provided, a user may navigate to this region to get a good overall view of building compliance with the relevant healthy building guidelines.

In some instances, and as optionally indicated at block 50, the method 40 may further include displaying one or more windows that instruct a user how to correct for a parameter that is outside of its corresponding healthy building range. In some instances, and as optionally indicated at block 52, the method 40 may further include modifying operation of one or more building system components in order to drive a parameter that is outside of its corresponding healthy building range towards a value that is within its corresponding healthy building range. While not expressly illustrated, it will be appreciated that the building 12 may include a variety of building systems such as but not limited to an HVAC system, and the HVAC system may include heating, cooling and/or ventilating components that may be used to alter a current value of a parameter (e.g. by changing a set point, opening a damper or valve, activating a fan, changing a fan speed, etc.). In some cases, the healthy building dashboard may also include a summary of healthy building alarms, wherein each health building alarm corresponds to a parameter in a zone (such as one of the zones 14) that falls outside or otherwise does not meet its corresponding healthy building range.

One of the plurality of different parameters for which a current parameter value may be compared to a corresponding healthy building range includes air temperature. The healthy building range for air temperature may correspond to a range of, for example, 68 to 74 degrees Fahrenheit. In some cases, air temperatures within this range can help to reduce the spread of disease within the building 12. It will be appreciated that these temperatures also generally correspond to those that are considered to be comfortable by a majority of people, although the upper temperature limit of 74 degrees Fahrenheit may be viewed as lower than historically preferred for energy savings, especially for summertime air conditioning.

Another of the plurality of different parameters for which a current parameter value may be compared to a corresponding healthy building range includes relative humidity. The healthy building range for relative humidity may correspond to, for example, a range of 40 to 60 percent relative humidity. In some cases, relative humidity values within this range can help to reduce the spread of disease within the building 12. In some cases, the next preferred relative humidity is in a range of 60 to 70 percent relative humidity. The next preferred relative humidity is a relative humidity in excess of 70 percent relative humidity. The next preferred is a relative humidity in a range of 30 to 50 percent relative humidity. A least preferred relative humidity is a relative humidity that is less than 30 percent relative humidity.

Another of the plurality of different parameters for which a current parameter value may be compared to a corresponding healthy building range includes carbon dioxide concentration. The healthy building range for carbon dioxide concentration may correspond to a carbon dioxide concentration of less than 800 parts per million (ppm). It will be appreciated that the primary source of carbon dioxide within the building 12 is people exhaling, and thus may be related to a density of occupants within the building. In some cases, a carbon dioxide concentration of less than 800 ppm can help to reduce the spread of disease within the building 12.

Additional examples of different parameters for which a current parameter value may be compared to a corresponding healthy building range include carbon monoxide concentration and total volatile organic compound (TVOC) concentration. The healthy building range for carbon monoxide concentration is less than 20 ppm. The healthy building range for TVOC concentration is less than 0.5 milligrams per cubic meter (mg/m³). It will be appreciated that carbon monoxide and TVOCs are both undesirable, and thus there is a desire to minimize (or even eliminate) detectable concentrations of either. In some cases, a carbon monoxide concentration of less than 20 ppm and/or a TVOC concentration of less than 0.5 mg/m³ can help to reduce the spread of disease within the building 12.

Some parameters for which a current parameter value may be compared to a corresponding healthy building range include parameters that are more behavior-based. An example of this is occupancy percentage. In accordance with healthy building guidelines, there may be a desire to limit relative occupancy of a space such as one or more of the zones 14 within the building 12. Limiting relative occupancy has the impact of increasing relative distances between people over time. While there may be instances in which a first person is too close to a second person, even transiently, it will be appreciated that having relatively fewer people in a particular space will tend to increase the relative distances between them. This can help with achieving social distancing. In some cases, a relative occupancy of less than 50 percent of a specified maximum occupancy may be desired. In some instances, a relative occupancy of less than 25 percent of a specified maximum occupancy may be desired. The specified maximum occupancy may be determined based on one or more characteristics of the particular space, and in some cases may represent the maximum safe allowed occupancy of that space as specified by the fire code of the municipality, state or the like in which the building 12 is located.

In some cases, the healthy building dashboard may include a summary of healthy building security parameters. These healthy building security parameters, which may be considered as additional examples of behavior-based parameters, may include one or more of an occupant temperature compliance parameter that relates to a status of occupant temperature compliance of occupants of the building. This can include an indication of whether occupants within the building have a healthy body temperature as opposed to an elevated body temperature that may be an indication of disease. Individual body temperatures may be estimated, for example, by taking infrared pictures of the individuals using an infrared security camera spaced throughout the building or performing a temperature screening at one or more access points of the building.

Another example of a healthy building security parameter is a mask compliance parameter that relates to a status of mask compliance of occupants of the building with one or more mask guidelines specified by the healthy building guidelines. If people are wearing masks during circumstances that warrant masks, this can help to reduce disease spread. Conversely, people who are not wearing masks during circumstances that warrant masks, this can worsen disease spread. Another example of a healthy building security parameter is a social distancing compliance parameter that relates to a status of social distancing compliance of occupants of the building with one or more social distancing guidelines specified by the healthy building guidelines. Another example of a healthy building security parameter is a maximum occupancy compliance parameter that relates to a status of maximum occupancy compliance of occupants of the building with one or more maximum occupancy guidelines specified by the healthy building guidelines. These are just examples, and additional healthy building security parameters are contemplated. Mask compliance, social distancing and maximum occupancy can each be identified by, for example, performing video analytics on video images captured by security camera spaced throughout the building.

In some cases, the healthy building dashboard may include a zone summary that for each of the plurality of different zones of the building which displays each current parameter value measured within that zone. The zone summary may, for example, highlight any current parameter value in any zone of the plurality of different zones that is currently outside of the corresponding healthy building range for that parameter. In some instances, the zone summary may include a recitation of the corresponding healthy building range for each of the parameters. In some cases, the zone summary may include links that when selected by a user display additional information. The additional information may include numerical values for one or more of the plurality of different parameters displayed over time to show trends. The additional information may include instructions as to how to improve the numerical values for one or more of the plurality of different parameters (e.g. change a set point, send a notification to individual occupants, add a sensor or other equipment such as a humidifier to the building facility management system, etc.). In some cases, the additional information may include a Standard Operating Procedure (SOE) that be define a set of steps that the operator should take to address the situation.

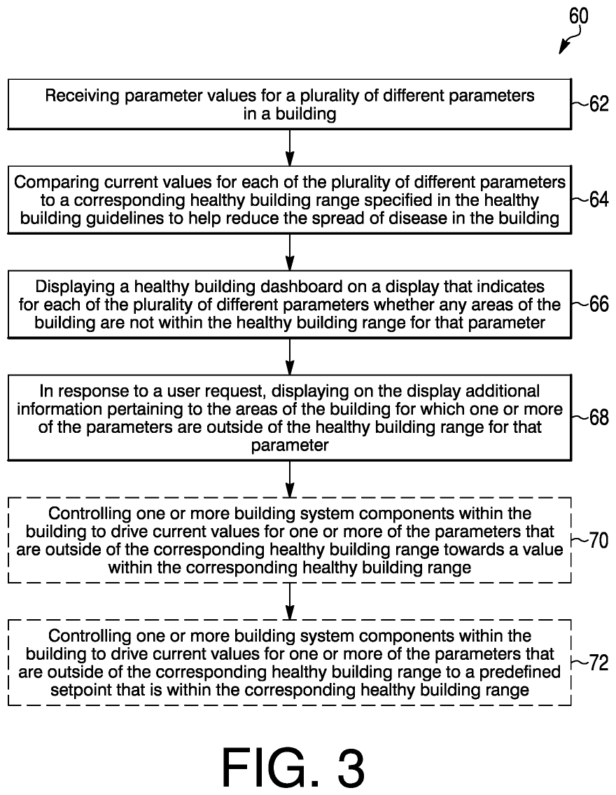

FIG. 3 is a flow diagram showing an illustrative method 60 of monitoring building compliance with healthy building guidelines, where the healthy building guidelines specify desired ranges for each of a plurality of different parameters. Parameter values for a plurality of different parameters in a building (such as the building 12) are received, as indicated at block 62. The current values for each of the plurality of different parameters are compared to a corresponding healthy building range specified in the healthy building guidelines to help reduce the spread of disease in the building, as indicated at block 64. A healthy building dashboard is displayed on a display that indicates for each of the plurality of different parameters whether any areas of the building are not within the healthy building range for that parameter, as indicate at block 66. In response to a user request, additional information is displayed on the display that pertains to the areas of the building for which one or more of the parameters are outside of the healthy building range for that parameter, as indicated at block 68.

In some instances, and as optionally indicated at block 70, the method 60 may further include controlling one or more building system components within the building to drive current values for one or more of the parameters that are outside of the corresponding healthy building range towards a value within the corresponding healthy building range (e.g. change a set point, send a notification to individual occupants, add a sensor or other equipment such as a humidifier to the building facility management system, etc.). In some cases, and as optionally indicated at block 72, the method 60 may further include controlling one or more building system components within the building to drive current values for one or more of the parameters that are outside of the corresponding healthy building range to a predefined setpoint that is within the corresponding healthy building range.

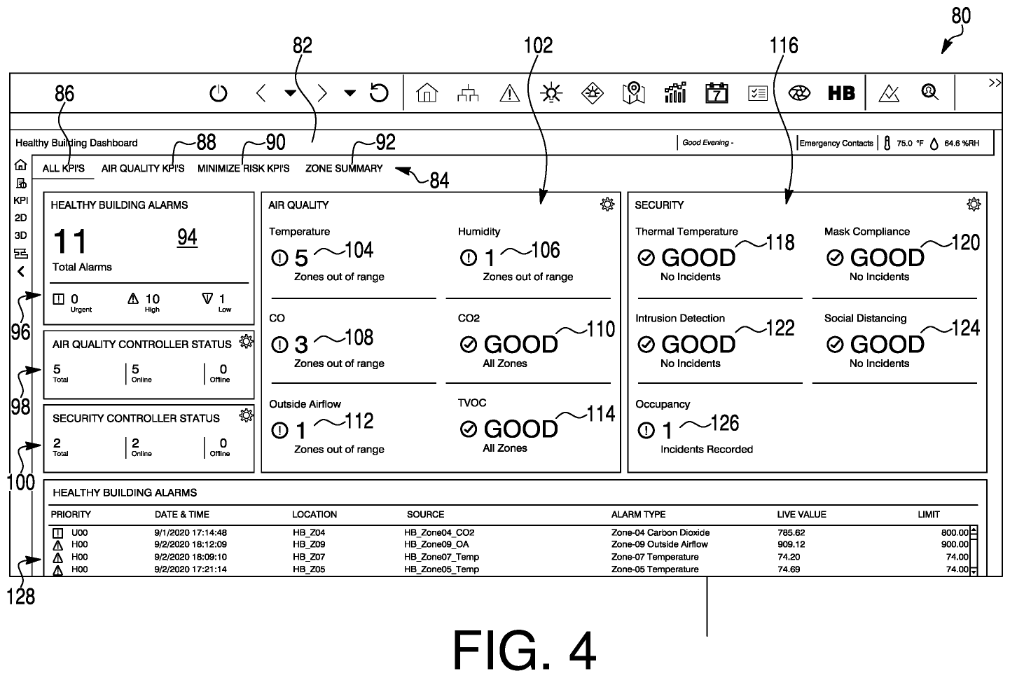

FIGS. 4 through 21 provide illustrative screen shots showing a variety of healthy building dashboards that may be displayed, for example, on the display 36 of the computing device 18. In these dashboards, it will be appreciated that the parameters are sometimes referred to as KPIs, or Key Performance Indicators. The dashboards described herein can for example display all KPIs, including Air Quality KPIs and Healthy Building Security KPIs. In this, Security KPIs are those parameters that refer to behavioral-related parameters such as body temperature, mask compliance, intrusion detection, social distancing and occupancy. The dashboards may display only Air Quality KPIs. The dashboards may display only Healthy Building KPIs. The dashboards may display only zone summaries. These are just examples.

FIG. 4 shows a screen 80 that includes a dashboard 82. The dashboard 82 includes a header 84 that includes an icon 86 for ALL KPIS, an icon 88 for AIR QUALITY KPIS, an icon 90 for MINIMIZE RISK KPIS (security KPIs) and a ZONE SUMMARY icon 92. As can be seen, the icon 86 for ALL KPIS is underlined or otherwise highlighted, indicating that the dashboard 82 is currently displaying all available KPIs. A user can tab between the icons 86, 88, 90, 92 included in the header 84 by clicking on or otherwise selecting a desired icon 86, 88, 90, 92. The dashboard 82 includes a HEALTHY BUILDING ALARMS panel 94 that provides summary information regarding current alarms. The HEALTHY BUILDING ALARMS panel 94 includes a summary section 96 that illustrates a current number of urgent alarms, a current number of high priority alarms and a current number of low priority alarms. The HEALTHY BUILDING ALARMS panel 94 also shows a total number of alarms. The HEALTHY BUILDING ALARMS panel 94 also includes an AIR QUALITY CONTROLLER STATUS section 98 that identifies how many air quality controllers are present and online. The HEALTHY BUILDING ALARMS panel 94 also includes a SECURITY CONTROLLER STATUS section 100 that identifies how many security controllers are present and online.

The illustrative dashboard 82 includes an AIR QUALITY panel 102 that indicates for each of a number of air quality parameters (or KPIs), whether the current values for these parameters within any of the zones are in range or out of range, along with a total number of zones that are in range or out of range. The AIR QUALITY panel 102 includes a temperature section 104, a humidity section 106, a CO section 108, a CO₂ section 110, an Outside Airflow section 112 and a TVOC section 114. As indicated, there are a total of five zones that are outside of the healthy building range for temperature, one zone that is outside of the healthy building range for humidity, three zones that are outside of the healthy building range for carbon monoxide and one zone that is outside of the healthy building range for outside air flow. Conversely, all reporting zones are within the healthy building range for carbon dioxide and for TVOC concentration.

The illustrative dashboard 82 includes a SECURITY panel 116 that indicates for each of a number of healthy building security parameters (or KPIs), whether the current values for these parameters within any of the zones are in range or out of range, along with the number of zones that are currently in violation of healthy building guidelines. The SECURITY panel 116 includes a thermal temperature section 118, a mask compliance section 120, an intrusion detection section 122, a social distancing section 124 and an occupancy section 126. As can be seen, there are currently no incidents reported for any of thermal temperature (meaning nobody has a detected body temperature in excess of a threshold), mask compliance (meaning all detected people are complying with mask guidelines, intrusion detection and social distancing (meaning all detected people are complying with interpersonal spacing guidelines). There is one zone that appears to be violating the occupancy guidelines, likely meaning that too many people are in a particular space or zone. This could also indicate a sensor problem, if for example, people are accurately detected entering the particular space or zone, but the people leaving the particular space or zone are not accurately detected leaving. This could result in a false positive.

The illustrative dashboard 82 includes an ALARMS detail section 128, that displays additional details regarding each of the alarms that were referenced in the HEALTHY BUILDING ALARMS panel 94. If there are more current alarms then there are available lines of text within the ALARMS detail section 128, a user is able to scroll up and down through the listed alarms. In some cases, alarms with a relatively higher priority may be listed at the top of the list, and alarms with a relatively lower priority may be listed at the bottom of the list.

It is contemplated that the illustrative dashboard 82 may collect and display only parameters that have a corresponding healthy building range specified by the healthy building guidelines, but this is not required. In some cases, all available parameters that have a corresponding healthy building range specified by the healthy building guidelines are displayed in the dashboard 82. When so provided, a user may navigate to this dashboard 82 to get a good overall view of building compliance with the relevant healthy building guidelines.

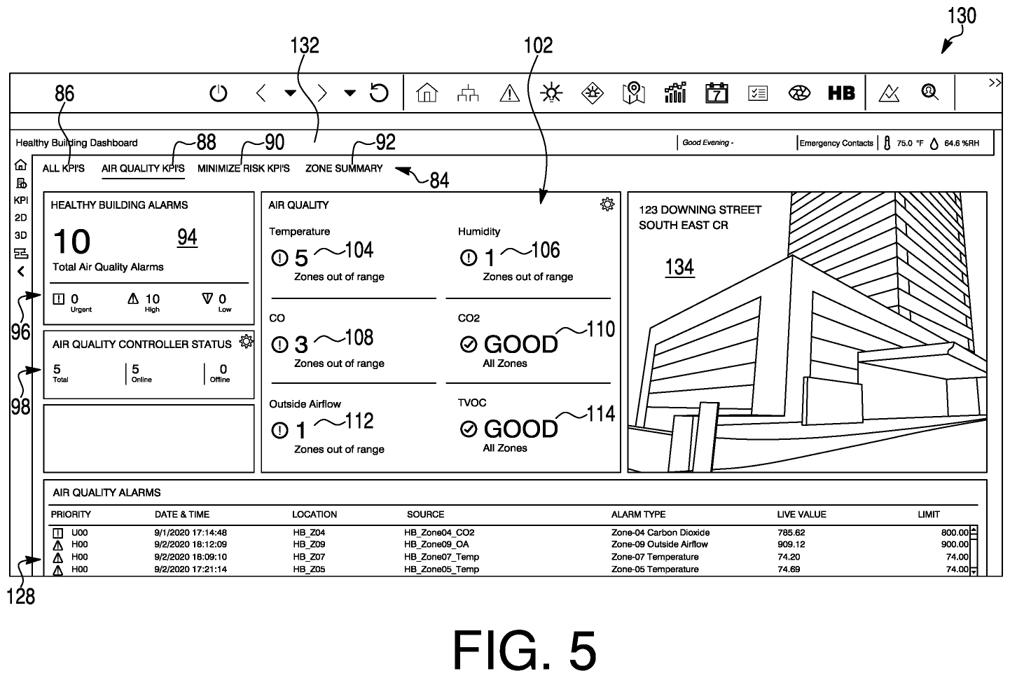

FIG. 5 shows a screen 130 that includes a dashboard 132. As can be seen from the header 84, the icon 88 for AIR QUALITY KPIS is underlined or otherwise highlighted, indicating that it has been selected. The dashboard 132 is similar to the dashboard 82, but does not display any information regarding security. Indeed, the SECURITY panel 116 shown in FIG. 4 has been replaced with a picture 134 of the building 12. This is merely representative, as any desired picture or other information can be displayed instead. In some cases, no picture 134 is displayed, and the remaining portions of the dashboard 132 are simply enlarged to fill up the available space. It should be noted that the HEALTHY BUILDING ALARMS panel 94 still includes the AIR QUALITY CONTROLLER STATUS section 98 that identifies how many air quality controllers are present and online but no longer includes the SECURITY CONTROLLER STATUS section 100 that identifies how many security controllers are present and online.

In comparing FIG. 5 to FIG. 4, it can be seen that summary section 96 that illustrates a current number of urgent alarms, a current number of high priority alarms and a current number of low priority alarms indicates a total of 11 alarms in FIG. 4 but only indicates a total of 10 alarms in FIG. 5. This is an indication that of the 11 alarms shown in total in FIG. 4, a total of 10 alarms are air quality related while only 1 alarm is security related.

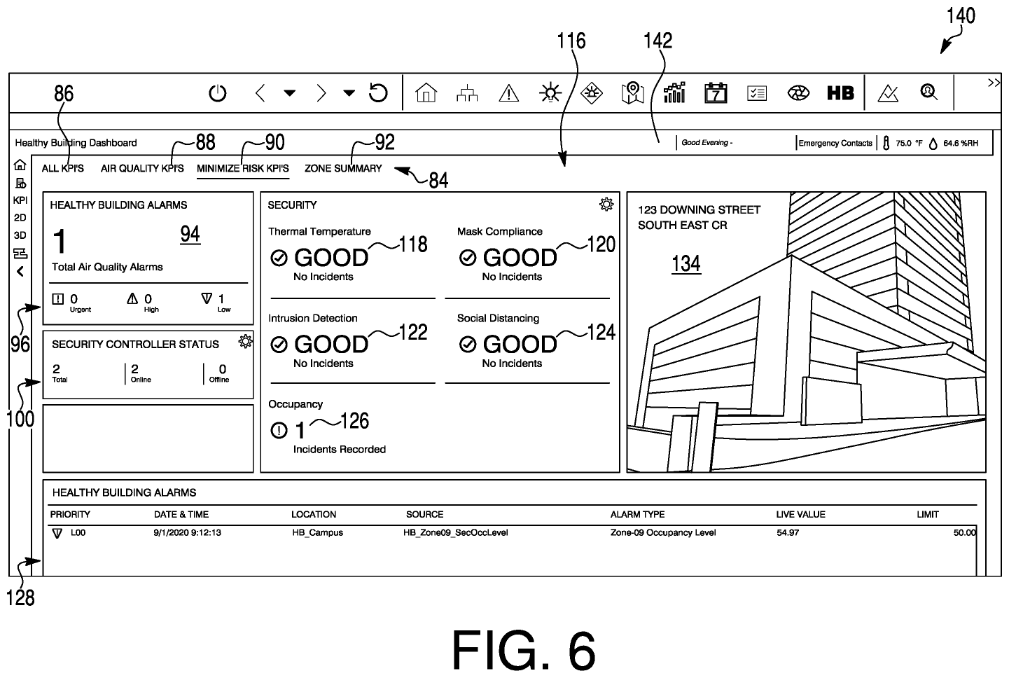

This can be seen in FIG. 6, which shows a screen 140 that includes a dashboard 142. As can be seen from the header 84, the icon 90 for MINIMIZE RISK KPIS is underlined or otherwise highlighted, indicating that it has been selected. In the summary section 96, it can be seen that there is only 1 alarm indicated. The HEALTHY BUILDING ALARMS panel 94 includes the SECURITY CONTROLLER STATUS section 100 that identifies how many security controllers are present and online, but does not include the AIR QUALITY CONTROLLER STATUS section 98 that identifies how many air quality controllers are present and online. The ALARMS detail section 128 can be seen as only including one alarm, which appears to be related to a detected occupancy level that has exceeded the healthy building range for that parameter.

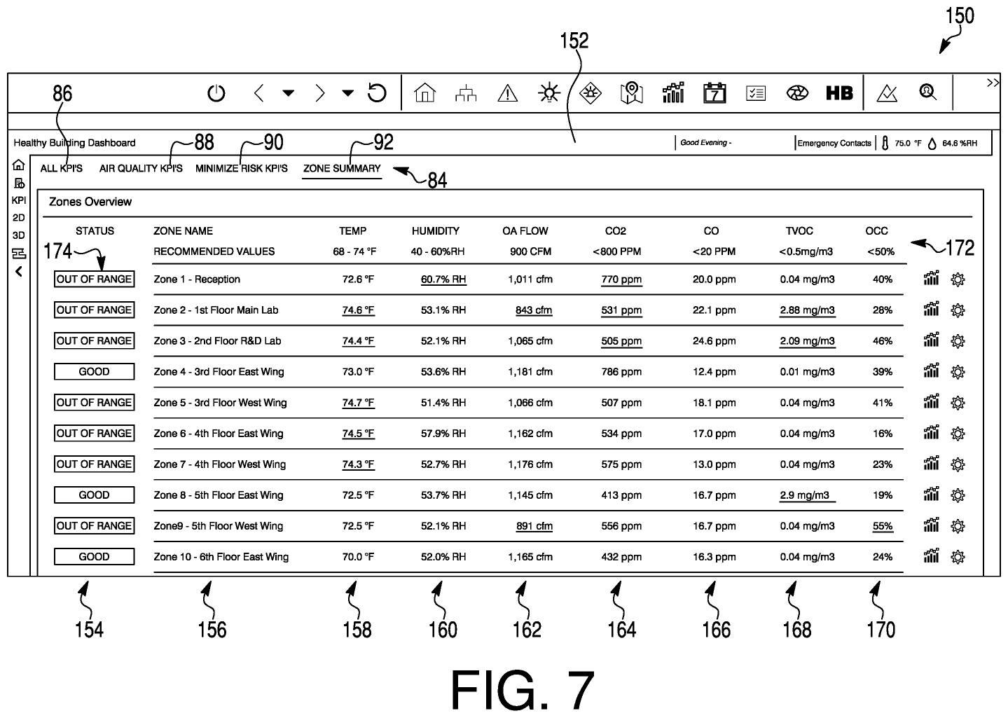

FIG. 7 shows a screen 150 that includes a dashboard 152. As can be seen from the header 84, the icon 92 for ZONE SUMMARY is underlined or otherwise highlighted, indicating that it has been selected. The dashboard 152 provides greater detail into the alarms that were illustrated in FIGS. 3 through 6. The dashboard 152 includes a status column 154, a zone name column 156, a temperature column 158, a humidity column 160, an OA flow column 162, a CO2 column 164, a CO column 166, a TVOC column 168 and an OC column 170. The dashboard 152 also includes a row 172 that illustrates the healthy building range for each of the displayed parameters.

For each zone that is listed, the status column 154 indicates whether that particular zone is fully in compliance with all of the healthy building guidelines or if one or more of the sensed parameter values is outside of the healthy building range for that particular parameter. The status column 154 may include an icon that says OUT OF RANGE for a particular zone if one or more parameters within that zone are out of their healthy building range. The status column 154 may include an icon that says GOOD for a particular zone if all of the parameters within that zone are within their healthy building ranges. It will be appreciated that other words or phrases may also be used. In some cases, the icons within the status column 154 that indicate whether a particular zone is good, or is out of range, may also use color to provide a quick indication. For example, the icon may be red if out of range, green if within range. Again, other colors may also be employed.

In some cases, any parameter value that is out of range may be displayed in a different color, or may otherwise be highlighted or indicated. For example, parameter values that are within range may be displayed as white numbers on a black screen, while parameter values that are out of range may be displayed as red numbers on the black screen. In some cases, parameter values that are out of range may be bolded, or highlighted in a color. In some instances, as illustrated for example in FIG. 7, the parameter values that are out of range may be underlined.

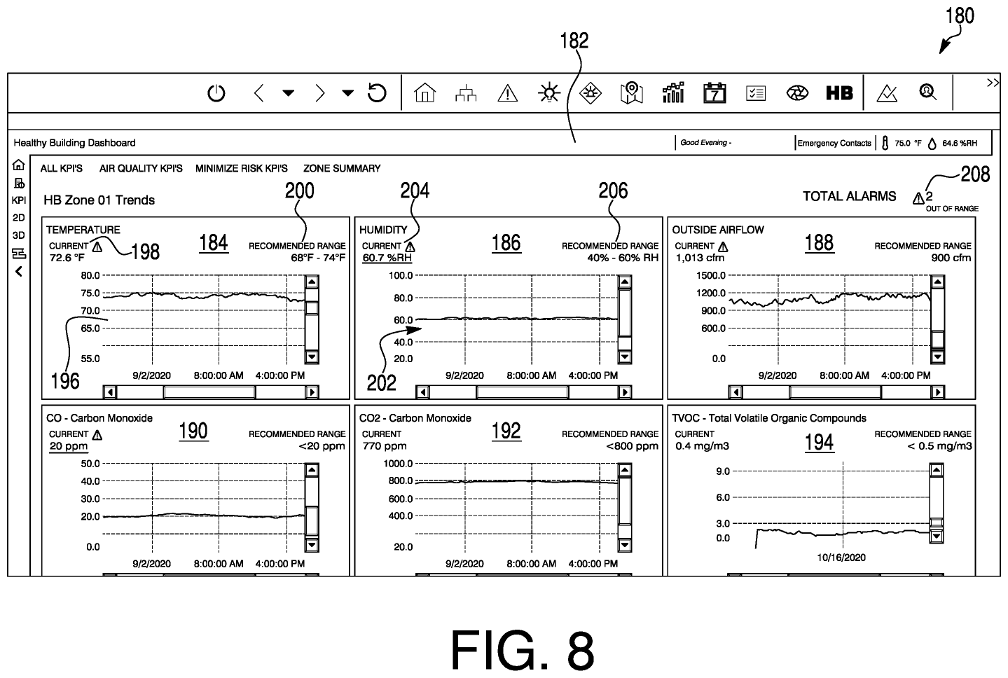

Clicking on one of the zones shown in FIG. 7 may result in additional numerical information being provided. As an example, if the “Zone 1—Reception” row 174 in FIG. 7 is selected, a screen 180 may be displayed as shown in FIG. 8. The screen 180 includes a dashboard 182. The dashboard 182 includes a graph showing trends in numerical data leading up to the currently displayed values. As shown, the dashboard 182 includes a temperature section 184, a humidity section 186, an outside airflow section 188, a CO section 190, a CO2 section 192 and a TVOC section 194. Each section displays a graph showing numerical trends for that parameter, a current measured value for that parameter and a recommended healthy building range for that parameter.

To illustrate, the temperature section 184 provides an example of a parameter value that is within range. The temperature section 184 includes a temperature graph 196 that shows how the measured temperature has been trending that day, a current temperature value 198 and a recommended range 200. As can be seen, the current temperature value of 72.6 degrees Fahrenheit is within the recommended range of 68 to 74 degrees Fahrenheit. Conversely, the humidity section 186 provides an example of a parameter value that is out of range. The humidity section 186 includes a humidity graph 202 that shows how the measured relative humidity has been trending that day, a current humidity value 204 and a recommended range 206. As can be seen, the current humidity value of 60.7 percent relative humidity exceeds the recommended range of 40 to 60 percent relative humidity.

The dashboard 182 includes an ALARMS summary 208 that shows that zone 1 has a total of 2 alarms. Looking at the dashboard 182, it can be seen that the two alarms correspond to humidity and carbon monoxide concentration. In some cases, the parameter values that are out of range may be displayed in a different color, bolded, highlighted or otherwise be displayed in a way that catches a user's attention. As illustrated, the humidity value and the carbon monoxide concentration are underlined.

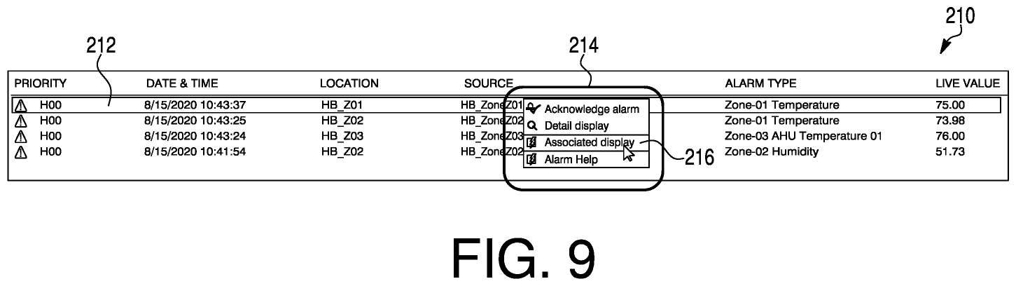

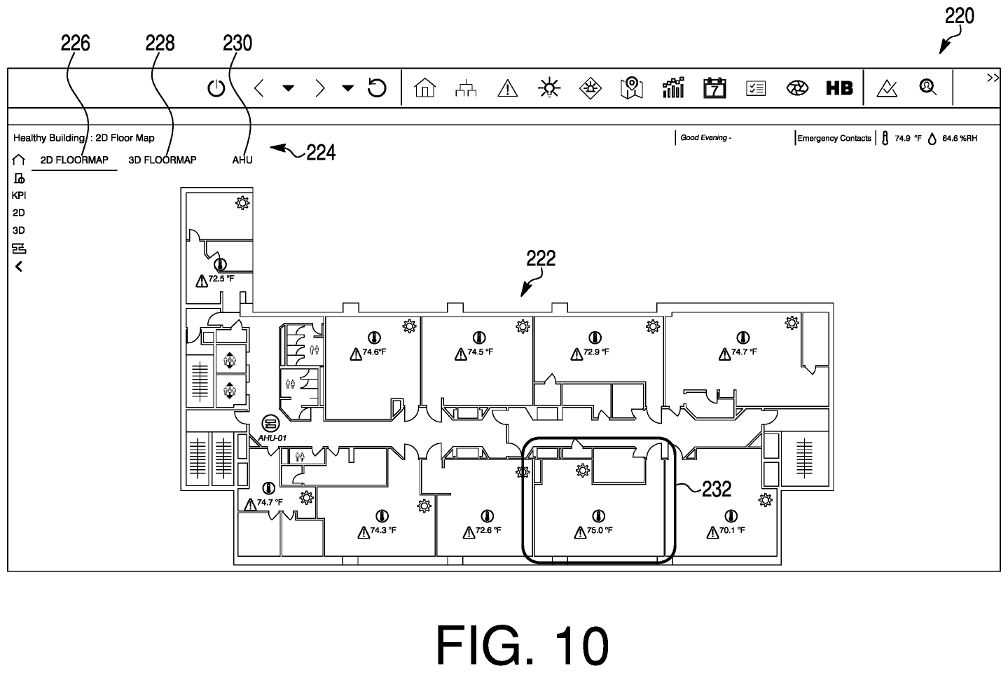

FIG. 9 shows a portion of a dashboard that includes an ALARMS detail section 210 that may be considered as being an example of the ALARMS detail section 128 shown in FIGS. 4 through 6. It can be seen that the ALARMS detail section 210 includes several rows, each listing a different alarm. An operator can click on a row in order to reach a pull-down menu to provide several options, such as but not limited acknowledging the alarm. In this example, a first row 212 has been selected. The first row 212 involves a temperature value that may be out of range. In this particular example, a current temperature of 75 degrees Fahrenheit violates the healthy building range of 68 to 74 degrees Fahrenheit. Clicking on the first row 212 causes a popup menu 214 to appear. Clicking on the associated display icon 216 causes display of a screen 220, as shown in FIG. 10.

FIG. 10 shows a screen 220. The screen 220 includes a header 224 that includes an icon 226 for 2D FLOORMAP, an icon 228 for 3D FLOORMAP and an icon 230 for AHU. As can be seen, the icon 226 for 2D FLOORMAP has been selected, and accordingly the screen is displaying a two-dimensional floor plan graphic 222 that includes a number of zones. Zone 1, which is causing the temperature alarm, is indicated at 232. In some cases, any zone that has a healthy building parameter that is in the healthy building range for that parameter may be displayed in one color while any zone that has a healthy building parameter that is outside of the healthy building range for that parameter may be displayed in another color. For example, green and red may be used, respectively.

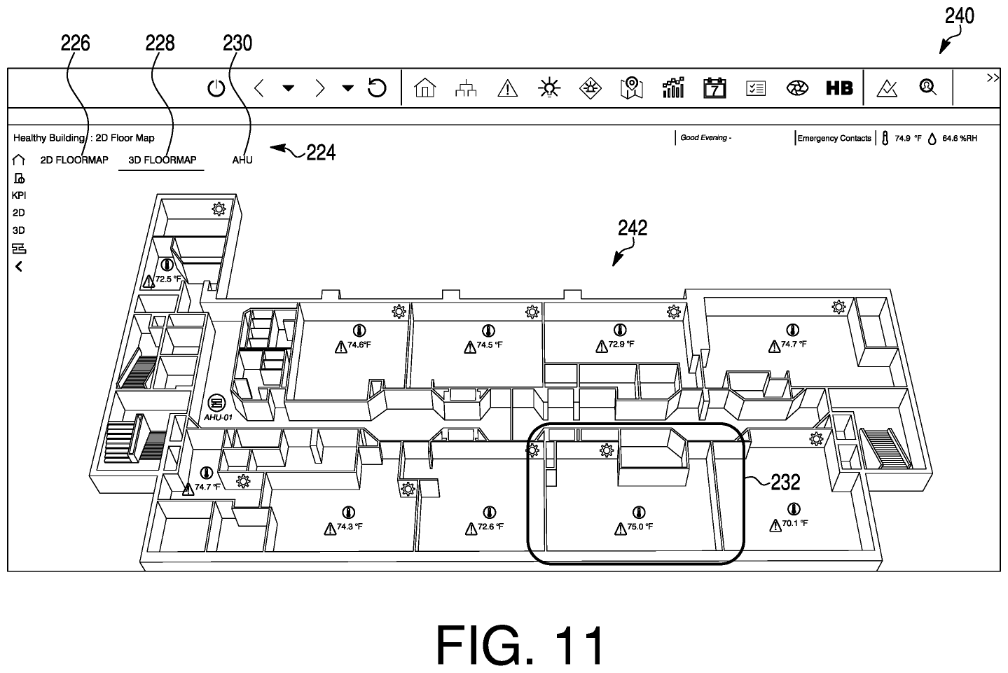

FIG. 11 shows a screen 240. As can be seen, the icon 228 for 3D FLOORMAP has been selected, and thus the screen 240 is displaying a three-dimensional floor plan graphic 242 that includes a number of zones. Zone 1, which is causing the temperature alarm, is indicated at 232. In some cases, any zone that has a healthy building parameter that is in the healthy building range for that parameter may be displayed in one color while any zone that has a healthy building parameter that is outside of the healthy building range for that parameter may be displayed in another color. For example, green and red may be used, respectively.

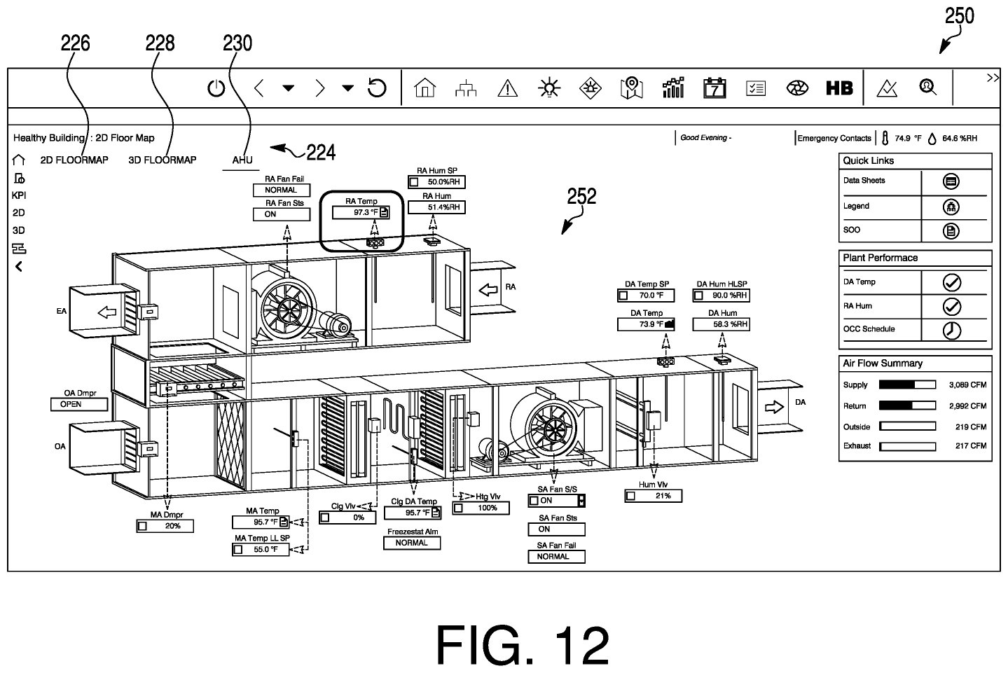

FIG. 12 shows a screen 250. As can be seen, the icon 230 for AHU has been selected, and thus the screen 250 is displaying a three dimensional graphic 252 showing the air handling equipment corresponding to the zone 1 that has a temperature value that is outside of the healthy building range. This allows a user to troubleshoot why the zone has a current temperature, or any of the other healthy building parameters, that is currently outside of the healthy building range for that particular healthy building parameter.

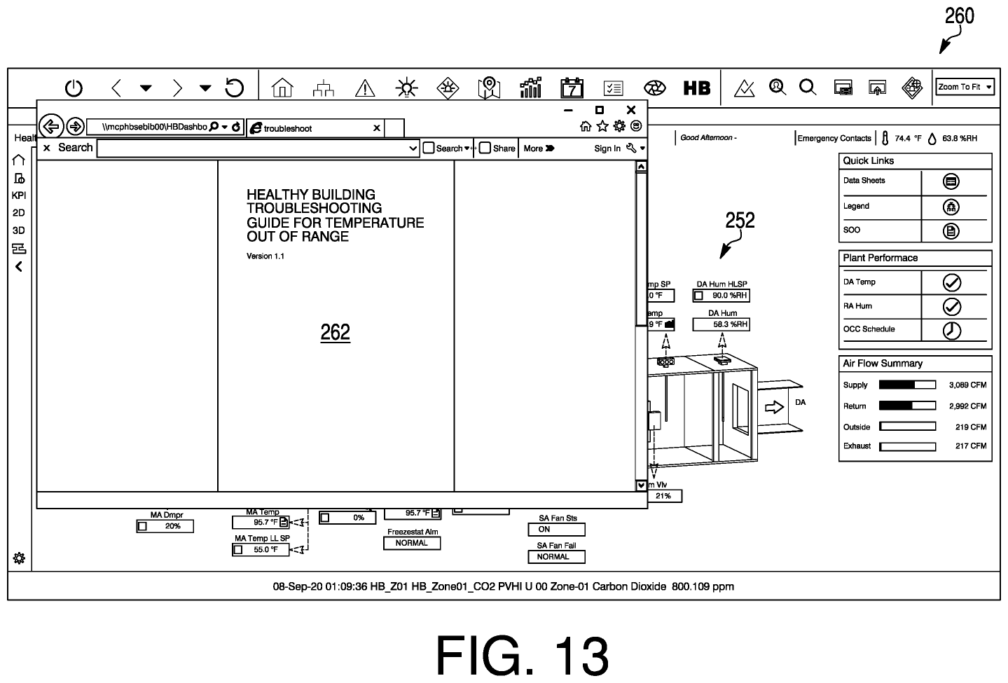

FIG. 13 shows a screen 260 that is similar to the screen 250 shown in FIG. 12, but includes a popup 262 that provides the user with additional information with how to resolve a healthy building parameter that is out of its healthy building range. As illustrated, the popup 262 includes documentation pertaining to troubleshooting a temperature that is out of range. The popup 262 may, for example, be a PDF format document that the user can scroll through. In some cases, the popup 262 may, for example, include a link to a video clip that guides the user through troubleshooting an out of range temperature value. The popup 262 may, for example, include a link to an appropriate Youtube.com link.

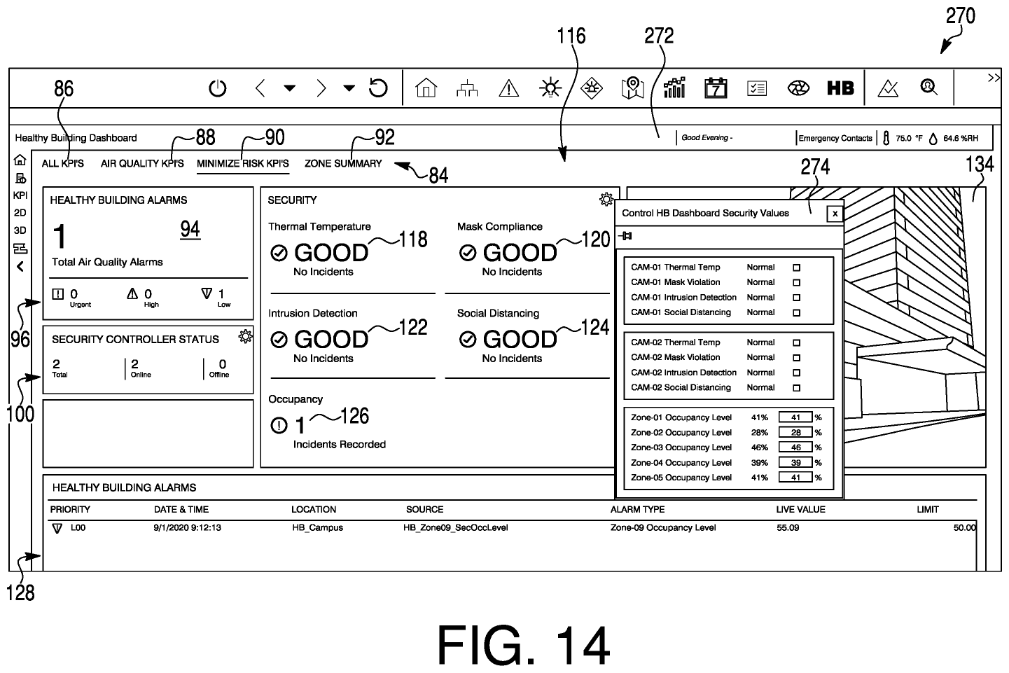

FIG. 14 shows a screen 270 that includes a dashboard 272. The dashboard 272 is similar to the dashboard 142 shown in FIG. 6, but includes a popup 274 that includes additional status data. In this particular example, the popup 274 includes details for CAM1 (Security Video Camera-1), including detected thermal temperatures, mask violation, intrusion detection and social distancing. Each of these can be determined via video analysis. The popup 274 also includes similar details for CAM2. The popup 274 also lists occupancy status for each zone.

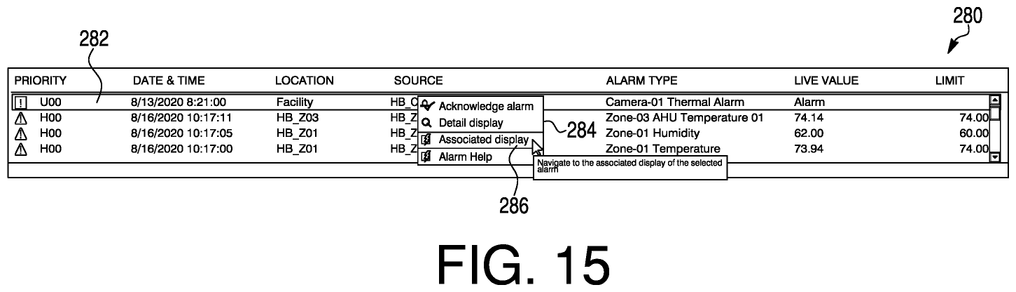

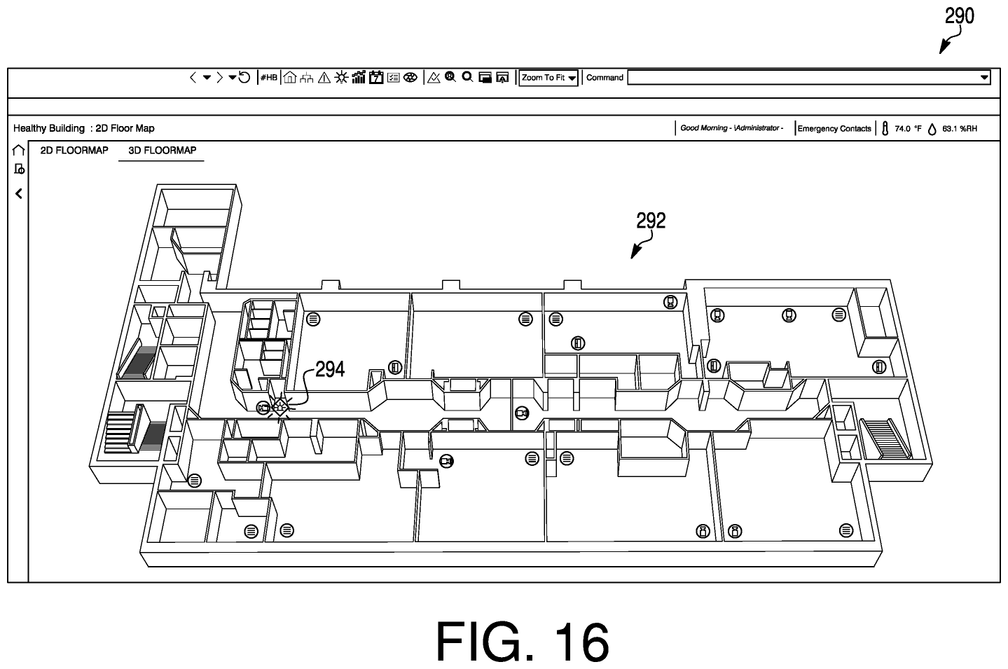

FIG. 15 shows a portion of a dashboard that includes an ALARMS detail section 280. It can be seen that the ALARMS detail section 280 includes several rows, each listing a different alarm. An operator can click on a row in order to reach a pull-down menu to provide several options, such as but not limited acknowledging the alarm. In this example, a first row 282 has been selected. The first row 282 involves a camera-indicated thermal alarm. This may indicate a person who has a camera-indicated body temperature that is high enough to trigger an alarm. Clicking on the first row 282 causes a popup menu 284 to appear. Clicking on the associated display icon 286 causes display of a screen 290, as shown in FIG. 16.

The screen 290 includes a three-dimensional floorplan graphic 292. The three-dimensional floorplan graphic 292 may include icons for cameras, doors, card readers, intercoms and the like. Clicking on a particular icon may pull up additional information. In FIG. 16, a camera icon 294 is highlighted. The camera icon 294 represents the particular camera that detected the possible thermal temperature violation. While the camera icon 294 is shown as highlighted, in some cases the camera icon 294 may be shown larger, or in a different color, than other camera icons present on the screen 290.

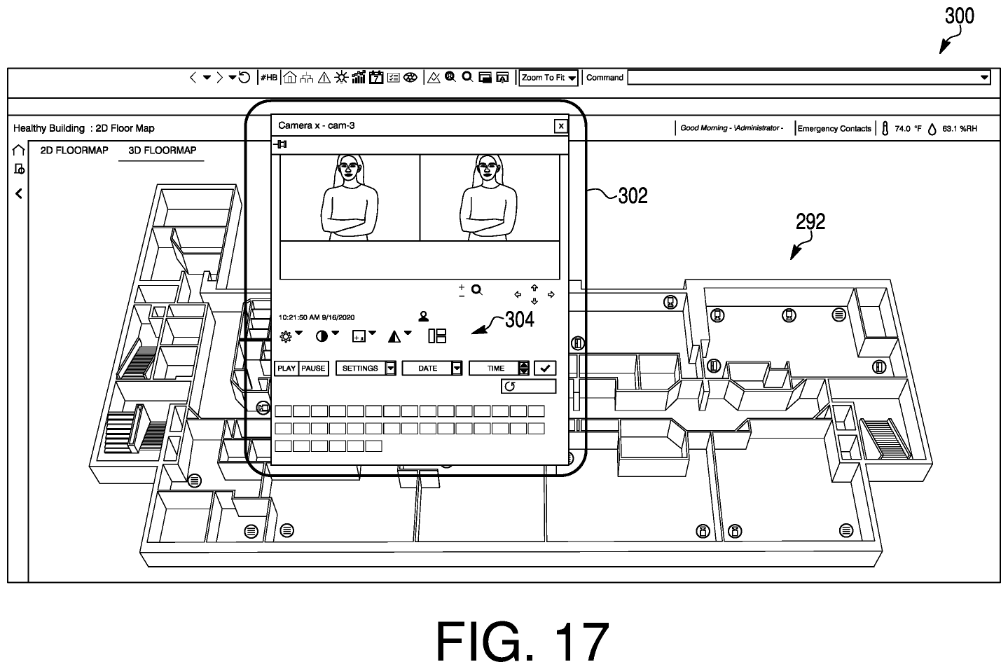

Clicking on the camera icon 294 will cause display of a screen 300, as shown in FIG. 17. The screen 300 includes a pop-up 302 that includes one or more images as captured by the camera represented by the camera icon 294. It will be appreciated that the images may be displayed in color, with different colors representing different detected skin temperatures. For example, blues may represent relatively lower temperatures while reds may represent relatively higher temperatures. This is merely illustrative, as any desired color scheme may be used. The pop-up 302 includes a set 304 of video controls, such as play, pause and the like, that a user may utilize to review video. The system may automatically identify the person in the video with the elevated skin temperature by using facial recognition or the like. In some cases, the persons contact information may be displayed in pop-up 302.

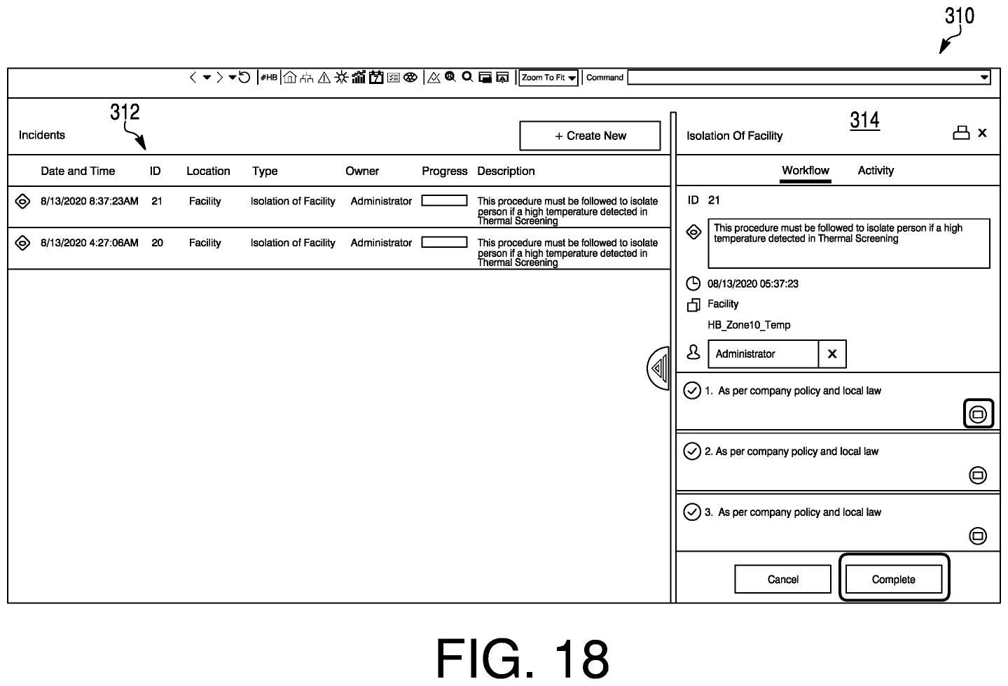

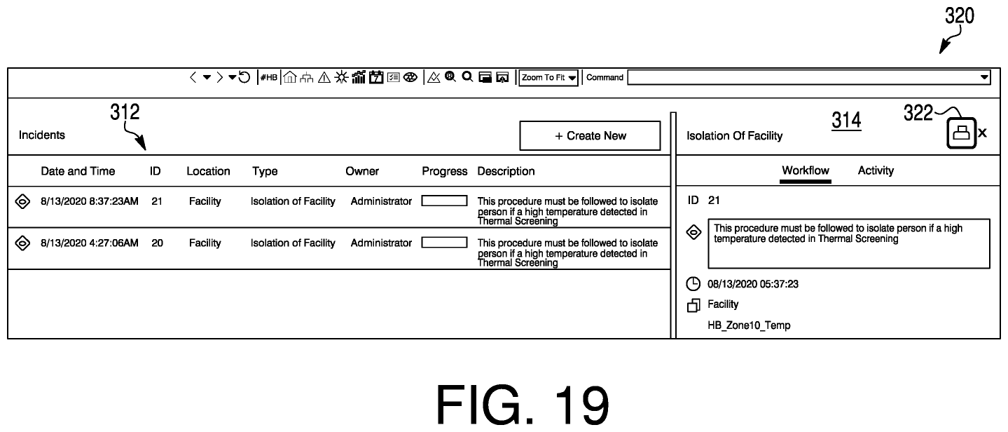

FIG. 18 shows a screen 310 that may be displayed in response to selecting an icon in FIG. 17. The screen 310 includes an Incident Workflow 312 that shows each open incident. Clicking on one of the workflows shown within the Incident Workflow 312 will open that incident on the right-side 314 of the screen 310. The Incident Workflow 312 may be a predefined Standard Operating Procedure (SOE) that defines a set of steps that the operator should take to address the corresponding incident. FIG. 19 shows a similar screen 320. Reports may be accessed by clicking a Report icon 322. While shown as a printer icon, it will be appreciated that other icons or graphics may also be used.

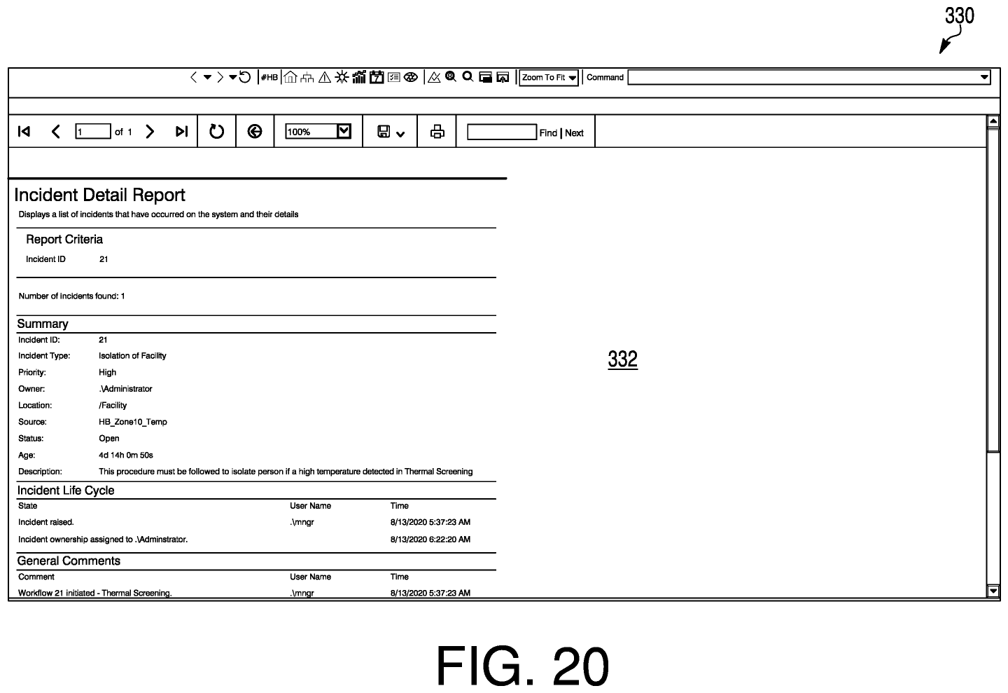

FIG. 20 shows a screen 330 that includes an incident report 332 that may be provided in response to the user clicking the Report icon 322 in FIG. 19. The incident report 332 may include all details including an alarm summary, incident life cycle and workflow action with operator details and date-time stamp.

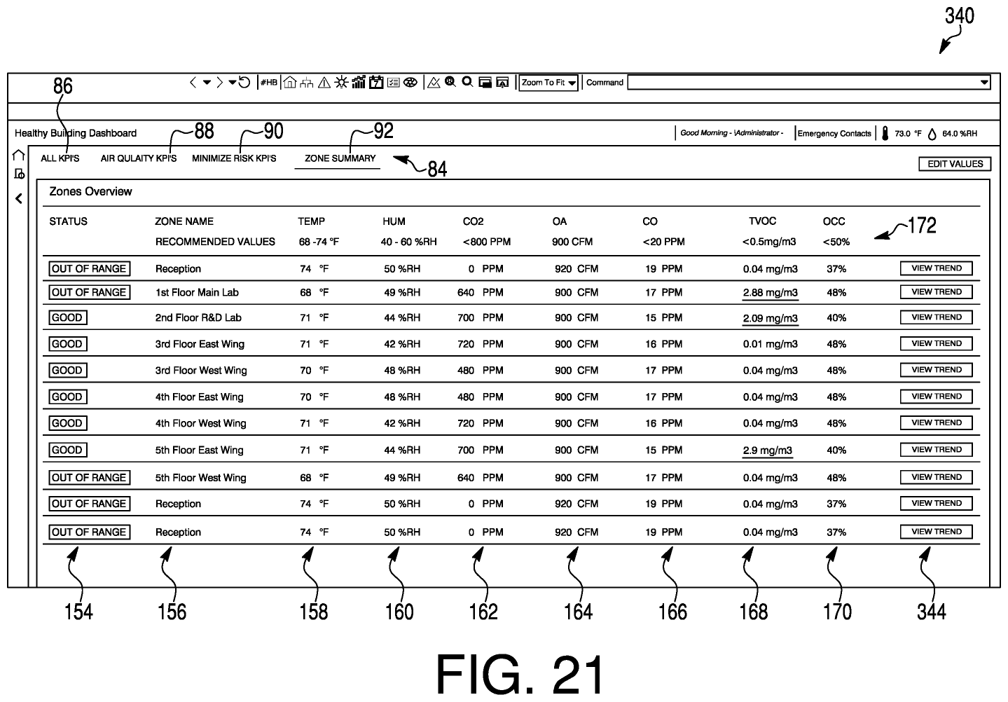

FIG. 21 shows a screen 340 that includes a dashboard 342. The dashboard 342 is similar to the dashboard 152 shown in FIG. 7, but includes a column 344 that includes for each row a VIEW TREND button. It will be appreciated that selecting the appropriate VIEW TREND button in the column 344 may cause display of a dashboard similar to the dashboard 182 shown in FIG. 8.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A method of monitoring building compliance with healthy building guidelines, the healthy building guidelines specifying a healthy building range for each of a plurality of different parameters, the method comprising:
obtaining current parameter values for the plurality of different parameters from a plurality of sensors disposed within a plurality of different zones of a building;
comparing each current parameter value received from each of the plurality of different zones with a corresponding healthy building range for that parameter as specified by the healthy building guidelines;
displaying a healthy building dashboard that includes a zone summary, wherein the zone summary:
displays, for each of the plurality of different zones, each current parameter value sensed within that zone;
highlights any current parameter value in any zone of the plurality of different zones that is currently outside of the healthy building range for that parameter; and
modifying operation of one or more building system components in order to drive a current parameter that is outside of its corresponding healthy building range towards a value that is within its corresponding healthy building range.

2. The method of claim 1, wherein the zone summary includes a recitation of the healthy building range for each of the plurality of different parameters.

3. The method of claim 1, wherein the zone summary includes one or more links that when selected by a user, display additional information including a graphical representation of one or more of the plurality of different parameters over period of time.

4. The method of claim 1, wherein the healthy building dashboard comprises one or more links that when selected by a user display instructions on how to correct one or more of the parameters that is outside of its corresponding healthy building range.

5. The method of claim 1, wherein the healthy building dashboard comprises a summary of healthy building alarms, wherein each health building alarm corresponds to a current parameter value falling within its corresponding healthy building range.

6. The method of claim 1, wherein one of the plurality of different parameters corresponds to air temperature, and its corresponding healthy building range is specified in the healthy building guidelines so as to help reduce the spread of disease in the building.

7. The method of claim 1, wherein one of the plurality of different parameters corresponds to relative humidity, and its corresponding healthy building range is specified in the healthy building guidelines so as to help reduce the spread of disease in the building.

8. The method of claim 1, wherein one of the plurality of different parameters corresponds to carbon dioxide concentration, and its corresponding healthy building range is specified in the healthy building guidelines so as to help reduce the spread of disease in the building.

9. The method of claim 1, wherein the plurality of different parameters comprises one or more of:
a carbon monoxide concentration;
a total volatile organic compound (TVOC) concentration; and
an occupancy percentage as a percent of a specified maximum occupancy.

10. The method of claim 1, wherein the healthy building dashboard comprises a summary of healthy building security parameters, wherein the healthy building security parameters include one or more of:
an occupant temperature compliance parameter that relates to a status of occupant temperature compliance of occupants of the building;
a mask compliance parameter that relates to a status of mask compliance of occupants of the building with one or more mask guidelines specified by the healthy building guidelines;
a social distancing compliance parameter that relates to a status of social distancing compliance of occupants of the building with one or more social distancing guidelines specified by the healthy building guidelines; and
a maximum occupancy compliance parameter that relates to a status of maximum occupancy compliance of occupants of the building with one or more maximum occupancy guidelines specified by the healthy building guidelines.

11. A method of monitoring compliance with healthy building guidelines, the healthy building guidelines specifying a healthy building range for each of a plurality of different sensed parameters, the method comprising:
receiving parameter values for a plurality of different sensed parameters in a building;
comparing current values for each of the plurality of different sensed parameters to a corresponding healthy building range specified in the healthy building guidelines to help reduce the spread of disease in the building;
displaying a healthy building dashboard on a display that indicates for each of the plurality of different sensed parameters whether any areas of the building are not within the healthy building range for that sensed parameter; and
controlling one or more building system components within the building to drive current values for one or more of the sensed parameters that are outside of the corresponding healthy building range towards a value within the corresponding healthy building range.

12. The method of claim 11, comprising controlling one or more building system components within the building to drive current values for one or more of the sensed parameters that are outside of the corresponding healthy building range to a predefined setpoint that is within the corresponding healthy building range.

13. The method of claim 11, wherein the healthy building dashboard indicates for each of the plurality of different sensed parameters how many areas of the building have that sensed parameter outside of the corresponding healthy building range.

14. The method of claim 11, wherein the healthy building dashboard indicates for each of the plurality of different sensed parameters which areas of the building have that sensed parameter outside of the corresponding healthy building range.

15. The method of claim 11, comprising
obtaining current parameter values for the plurality of different sensed parameters from a plurality of sensors disposed within a plurality of different areas of a building;
comparing each current sensed parameter value received from each of the plurality of different areas with a corresponding healthy building range for that parameter as specified by the healthy building guidelines; and
displaying an area summary, wherein the area summary displays, for each of the plurality of different areas, each current sensed parameter value measured within that area.

16. The method of claim 15, further comprising, in the area summary, highlighting any current parameter value in any area of the plurality of different areas that is currently outside of the healthy building range for that parameter.

17. The method of claim 15, wherein the area summary includes a recitation of the healthy building range for each of the sensed parameters.

18. The method of claim 11, wherein the healthy building dashboard includes one or more links that when selected by a user display additional information including a graphical representation of one or more of the plurality of different sensed parameters over a period of time.

19. A method of monitoring building compliance with healthy building guidelines, the healthy building guidelines specifying a healthy building range for each of a plurality of different parameters, the method comprising:
obtaining current parameter values for a plurality of different parameters from a plurality of sensors disposed within a plurality of different zones of a building;
comparing each current parameter value received from each of the plurality of different zones with a corresponding healthy building range for that parameter as specified by the healthy building guidelines;
displaying a healthy building dashboard that displays, for each of the plurality of different parameters, a quantity of the plurality of zones of the building that are not within the healthy building range for that parameter;
the healthy building dashboard including a link, that when selected by user, displays a zone summary, wherein the zone summary displays a table that includes the current parameter value of each of a plurality of different parameters in each of the plurality of zones of the building; and
modifying operation of one or more building system components in order to drive a current parameter that is outside of its corresponding healthy building range towards a value that is within its corresponding healthy building range.

20. The method of claim 19, further comprising highlighting in the table any current parameter value that is currently outside of the healthy building range for that parameter.
